(19) United States
(12) Patent Application Publication
(10) Pub. No.: US 2004/0068006 A1
(43) Pub. Date: Apr. 8, 2004

(54) Method for preventing acute renal failure

(75) Inventors: Mitchell P. Fink, Pittsburgh, PA (US); Howland Shaw Warren JR., Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Critical Therapeutics, Inc., Cambridge, MA (US)

(21) Appl. No.: 10/679,040

(22) Filed: Oct. 3, 2003

Related U.S. Application Data
(63) Continuation-in-part of application No. PCT/US02/10539, filed on Apr. 3, 2002.
(60) Provisional application No. 60/281,363, filed on Apr. 4, 2001.

(51) Int. Cl. A61K 31/22; A61K 31/16

(52) U.S. Cl. 514/546; 514/625

(57) ABSTRACT

Disclosed is a method of treating acute renal failure in a subject. The method comprises the step of administering to the subject an effective amount of a composition comprising a 2-ketoalkanoic acid, a pharmaceutically acceptable salt of a 2-ketoalkanoic acid, an ester of a 2-ketoalkanoic acid, or an amide of a 2-ketoalkanoic acid. Preferably, the composition comprises an enolization agent and an alkyl aralkyl, alkoxyalkyl or carboxyalkyl ester of a 2-ketoalkanoic acid dissolved in a pharmaceutically acceptable vehicle.

### RELATED APPLICATION

This application is a continuation-in-part of International Application No. PCT/US02/10539, which designated the United States and was filed Apr. 3, 2002, published in English, which claims the benefit of U.S. Provisional Application No. 60/281,363, filed on Apr. 4, 2001. The entire teachings of these applications are incorporated herein by reference.

### BACKGROUND OF THE INVENTION

Renal failure is a major cause of long-term hospitalization and death. It is characterized by acute or chronic deterioration of kidney function that initially occurs in an individual who previously had normal kidney function or that progresses further in an individual already suffering from kidney disease and/or dysfunction. There are a number of factors which are predictive of whether a patient is likely to experience acute renal failure. Risk factors include pre-existing diseases or conditions such as diabetes, renal disease/dysfunction, hypotension, hemorrhagic shock, systemic inflammation, sepsis, temporary interruption of blood flow to the kidneys, liver disease or heart disease. Other risk factors include treatment with nephrotoxic drugs and contrast imaging agents. Subjects with two or more risk factors are said to be “at risk” for acute renal failure.

Although it is now possible to identify patients who are at risk for developing acute renal failure, treatments for preventing the condition are still inadequate. Thus, there is an urgent need for new methods of preventing and/or ameliorating the effects of acute renal failure.

### SUMMARY OF THE INVENTION

It has now been found that ethyl pyruvate inhibits acute renal failure in a rat model (see Example 1). Based on this discovery, methods of treating acute renal failure by administering an ester or amide of a 2-ketoalkanoic acid are disclosed herein.

One embodiment of the present invention is a method of treating acute renal failure in a subject. The method comprises the step of administering to the subject an effective amount of a composition comprising a 2-ketoalkanoic acid, a pharmaceutically acceptable salt of a 2-ketoalkanoic acid, an ester of a 2-ketoalkanoic acid, or an amide of a 2-ketoalkanoic acid. Preferably, the composition comprises an enolization agent and an alkyl, aralkyl, alkoxyalkyl or carboxyalkyl ester of a 2-ketoalkanoic acid (preferably an ester of pyruvate such as ethyl pyruvate) dissolved in a pharmaceutically acceptable vehicle.

Another embodiment of the present invention is a method of prophylactically treating acute renal failure in a subject undergoing contrast imaging (preferably prior to the procedure). The method comprises the step of administering to the subject an effective amount of a composition comprising an alkyl aralkyl, alkoxyalkyl or carboxyalkyl ester of a 2-ketoalkanoic acid (preferably an ester of pyruvate such as ethyl pyruvate) dissolved in a pharmaceutically acceptable carrier vehicle. Preferably the composition additionally comprises an enolization agent.

### BRIEF DESCRIPTION OF THE FIGURES

FIG. 1 is a graph showing the increase in serum creatine levels in mg/dl over time in hours after antibiotic and volume resuscitated cecal ligation puncture. Serum creatine was assayed by a picric acid-based colorimetric kinetic autoanalyzer. P<0.05 vs. 0 hr.

FIG. 2 is a graph showing the increase in blood urea nitrogen in mg/dl over time in hours after antibiotic and volume resuscitated cecal ligation puncture sepsis in a mouse model. Serum creatine was assayed by HPLC. P<0.05 vs. 0 hr.

FIG. 3 is a graph showing the increase in serum creatine levels in mg/dl over time in hours after antibiotic and volume resuscitated cecal ligation puncture sepsis in a mouse model. Serum creatine was assayed by HPLC. P<0.05 vs. 0 hr.

FIG. 4 is a graph showing the increase in tubular damage score over time in hours after antibiotic and volume resuscitated cecal ligation puncture sepsis in a mouse model. The graphs show the tubular damage scores in the cortex or the outer stripe of the outer medulla (OSOM). Values are mean±SE (n=5-6 per group); P<0.05 vs. sham.

FIG. 5 is a graph showing the increase in blood urea nitrogen (mg/dl), serum creatine (mg/dl) and tubular damage score (cortex or outer stripe medulla (OSOM)) over time in hours after antibiotic and volume resuscitated cecal ligation puncture sepsis in a mouse model. Animals were untreated (sham), treated with a single dose of Ringers Lactate vehicle (RL) or a single dose of 8 or 40 mg/kg ethyl pyruvate at 0, 6 or 12 hours after cecal ligation puncture. P<0.05 vs. sham; P<0.05 vs. RL.

### DETAILED DESCRIPTION OF THE INVENTION

The present invention is directed to a method of treating acute renal failure in subject by administering a pharmaceutical composition comprising a 2-ketoalkanoic acid, a pharmaceutically acceptable salt of a 2-ketoalkanoic acid, an ester of a 2-ketoalkanoic acid, or an amide of a 2-ketoalkanoic acid. In one aspect, the composition comprises an alkyl, aralkyl, alkoxyalkyl or carboxyalkyl ester of a 2-ketoalkanoic acid dissolved in a pharmaceutically acceptable vehicle and optionally including an enolization agent. Acetoxyalkyl and carbalkoxy alkyl esters are also included.

In other aspects, the ester portion of the 2-ketoalkanoic acid ester is ethyl, propyl, butyl, carboxymethyl, acetoxymethyl, carbethoxymethyl or ethoxymethyl. Other specific examples include carbmethoxymethyl (—CH₂COOCH₃), carbmethoxyethyl (—CH₂CH₂COOCH₃), carbethoxyethyl (—CH₂CH₂COOCH₂CH₃) and methoxymethyl (—CH₂OCH₃) esters. Yet other groups suitable for esterification of 2-ketoalkanoic acids include: 1) dihydroxyacetone esters of formula:

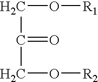

wherein R₁ is a 2-ketoalkanoate group such as pyruvyl and R₂ is H, a 2-ketoalkanoate group such as pyruvyl or a C1-C3 acyl group such as acetyl or propionyl; and 2) monosaccharide esters such as ribosyl and glucosyl esters:

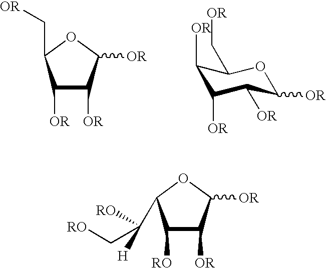

wherein each R is independently H, a 2-ketoalkanoate group such as pyruvyl or a C1-C3 acyl group such as acetyl or propionyl, provided that at least one R is an 2-ketoalkanoate group.

Examples of the 2-ketoalkanoic acid portion is 2-keto-butyrate, 2-ketopentanoate, 2-keto-3-methyl-butyrate, 2-keto-4-methyl-pentanoate or 2-keto-hexanoate. Specific examples of 2-ketoalkanoic esters suitable for use in the disclosed method include ethyl pyruvate, propyl pyruvate, carbmethoxymethyl pyruvate, acetoxymethyl pyruvate, carbethoxymethymethyl pyruvate, ethoxymethyl pyruvate, ethyl 2-ketobutyrate, ethyl 2-ketopentanoate, ethyl 2-keto-3-methyl-butyrate, ethyl 2-keto-4-methyl-pentanoate, or ethyl 2-keto-hexanoate. In a preferred embodiment, the pharmaceutical composition used in the disclosed method comprises ethyl pyruvate.

Suitable amides of 2-ketoalkanoic acids for use in the method of the present inventions include compounds having the following structural formula: RCOCONR1R2. R is an alkyl group; R1 and R2 are independently —H, alkyl, aralkyl, alkoxyalkyl, carboxyalkyl or —CHR3COOH; and R3 is the side chain of a naturally occurring amino acid.

Suitable alkyl groups include C1-C8 straight chained or branched alkyl group, preferably C1-C6 straight chained alkyl groups.

Suitable aryl groups include carbocyclic (e.g., phenyl and naphthyl) and heterocyclic (e.g., furanyl and thiophenyl) aromatic groups, preferably phenyl.

An alkoxy group is —OR4, wherein R4 is an alkyl group, as defined above. An alkoxyalkyl group is an alkyl group substituted with —OR4.

An aralkyl group is —XY, wherein X is an alkyl group and Y is an aryl group, both as defined above.

A carboxyalkyl group is an alkyl group substituted with —COOH. A carbalkoxyalkyl group is an alkyl group substituted with —C(O)OR, wherein R is an alkyl group, as defined above.

An acetoxy alkyl group is an alkyl group substituted with —O—C(O)—R, wherein R is an alkyl group, as defined above.

An “enolization agent” is a chemical agent which induces and stabilizes the enol resonance form of a 2-ketoester at or around physiological pH (e.g., between about 7.0 to about 8.0). Enolization agents include a cationic material, preferably a divalent cation such as calcium or magnesium or, for example, a cationic amino acid such omithine or lysine. The enolization agent in the composition of the invention is at an appropriate concentration to induce enolization of the 2-keto functionality of the amount of active ester agent in the administered composition, e.g., from 0.0 to 4.0 molar equivalents relative to the ester.

Formulation of a therapeutic agent to be administered will vary according to the route of administration selected (e.g., solution, emulsion, capsule). An appropriate composition comprising the agent to be administered can be prepared in a physiologically or pharmaceutically acceptable vehicle or carrier. A physiologically or pharmaceutically acceptable carrier for the composition used in the method of the present invention can be any carrier vehicle generally recognized as safe for administering a therapeutic agent to a mammal, e.g., a buffer solution for infusion or bolus injection, a tablet for oral administration or in gel, micelle or liposome form for on-site delivery. A preferred buffer solution is water or isotonic or hypertonic saline buffered with bicarbonate, phosphate, lactate or citrate at 0.1 M to 0.2 M. Alternatively, the therapeutic agent is administered in a plasma extender, microcolloid or microcrystalline solution. One preferred carrier is Ringer's isotonic saline solution comprising from about 105 mM to 110 mM NaCl, from about 3.8 mM to about 4.2 mM KCl and from about 2.5 to 2.9 mM CaCl₂. More preferably, the carrier is Ringer's Lactate solution comprising NaCl (preferably from about 105 mM to 110 mM), KCl (preferably from about 3.8 mM to about 4.2 mM), a lactate salt such as sodium lactate (preferably from about 25 mM to about 30 mM) and optionally CaCl₂ (preferably from about 2.5 to 2.9 mM). Preferably, acidity of the formulation is adjusted to a pH range of about 4 to about 8, even more preferably to a pH value of about 5 to about 7. Other carriers for the compounds of the present invention include isotonic salt solutions buffered with citrate, for example, approximately 100 mM to 200 mM citrate.

A preferred concentration range of the therapeutic agent is from about 0.1 to about 10% by weight. In a particularly preferred aspect, the pharmaceutical composition comprises approximately 10 mg/ml of ethyl pyruvate. A preferred example of the formulation used for treating renal failure comprises 2% to 3% ethyl pyruvate by weight, approximately 100 mM citrate buffer (or about 25 mM to about 30 mM of sodium lactate), about 4 mM KCl and, optionally, 2.7 mM CaCl₂. The formulation administered for the treatment of acute renal failure can be formed by admixing components of a two part formulation, one part containing, for example, ethyl pyruvate (neat), and the second part consisting of the remaining components of a desired aqueous formulation, for example, those reagents described above.

The therapeutic compositions of the invention can be administered orally, or parenterally, (e.g., intranasally, subcutaneously, intramuscularly, intravenously, intraluminally, intra-arterially, intravaginally, transurethrally or rectally) by routine methods in pharmaceutically acceptable inert carrier substances. For example, the therapeutic compositions can be administered in a sustained release formulation using a biodegradable biocompatible polymer, or by on-site delivery using micelles, gels, liposomes, or a buffer solution. Preferably, the pharmaceutical composition is administered as an infusate at a concentration of, e.g., 20-200 mM of 2-ketoalkanoic acid, at a rate of 10-100 mg/kg/hr, in a buffer solution as described herein. In bolus form, the active agent can be administered at a similar dosage, e.g., 1 mg/kg body weight/day to 200 mg/kg body weight/day of active agent, where the dosage is divided into aliquots and delivered 1 to 4 times daily (for a total dosage of 1 mg/kg body weight/day to 200 mg/kg body weight/day), with the concentration of the active agent adjusted accordingly. Optimal dosage and modes of administration can readily be determined by conventional protocols. Optimal dosage and modes of administration can readily be determined by conventional protocols.

The method of the present invention can be used to treat acute renal failure in subjects. It is particularly suited for prophylactic treatment of acute renal failure. “Prophylactic treatment” refers to treatment before kidney function has been adversely affected by a given disease or condition to prevent or reduce the extent of damage to renal function. For example, a subject at risk for acute renal failure can be prophylactically treated according to the method of the present invention prior to undergoing a contrast imaging procedure. “Prophylactic treatment” also refers to treatment after renal function has already been adversely affected by a given disease or condition to prevent or reduce further deterioration of renal function. For example, a subject at risk for acute renal failure who becomes septic or goes into hemorrhagic shock may suffer kidney damage before treatment can begin. However, treatment that is initiated after kidney damage has already occurred according to the method of the present invention can prevent further deterioration of kidney function.

A “subject” is preferably a human patient, but can also be a companion animal (e.g., dog, cat and the like), a farm animal (e.g., horse, cow, sheep, and the like) or laboratory animal (e.g., rat, mouse, guinea pig, and the like). The method of the present invention is ideally suited to prophylactically treat subjects at risk for acute renal failure, which includes subjects having more than one risk factor for the condition, e.g., two, three, four or more risk factors. Examples of risk factors include pre-existing diseases or conditions such as diabetes, renal disease/dysfunction, hypotension, hemorrhagic shock, systemic inflammation, sepsis, temporary interruption of blood flow to the kidneys, liver disease or heart disease. Other risk factors include treatment with nephrotoxic drugs and contrast imaging agents. The risk of suffering acute renal failure increases as the number of risk factors increases.

The invention is illustrated by the following example which is not intended to be limiting in any way.

### EXEMPLIFICATION

### Example 1

### Ethyl Pyruvate Decreases Sepsis Induced Acute Renal Failure in a Mouse Model

Animal care followed NIH criteria for the care and use of laboratory animals in research. Young (7-8 weeks) and aged (42-44 weeks) male C57BL/6 mice [National Institutes of Health (NIH), Frederick, Md.] had free access to water and chow (NIH-07) Rodent chow, Zeigler Bros., Gardners, Pa.) before and after surgery.

Aged mice were anesthetized with 100 mg/kg ketamine, 10 mg/kg xylazine, and 1 mg/kg acepromazine IM. After laparotomy, a 5-0 silk ligature was placed 5 mm from the cecal tip. The cecum was punctured twice with a 21-gauge needle and gently squeezed to express a 1 mm column of fecal material. In sham operated animals, the cecum was located, but neither ligated nor punctured. The abdominal incision was closed in two layers with 6-0 nylon sutures. After surgery, one ml of pre-warmed normal saline was given IP. All animals received a broad spectrum antibiotic (imipenem/cilastatin; 25 mg/kg SC), then 1.5 ml of 3/4 saline was administered at 6 and 18 hr after surgery by SC injection. At the time of sacrifice, blood was collected from abdominal aorta for measurement of blood chemistries. The kidneys were harvested for histological and mechanistic studies.

Survival after surgery was assessed every 6 hr within the first 48 hr and then every 8 hr for 4 days. Antibiotic injection and fluid resuscitation were started 6 hr after CLP by SC injection, and then repeated every 12 hr for 4 days.

Animals received 0.4 ml of Ringers lactate (RL) [130 mM Na+, 4 mM K+, 2.7 mM Ca+, 109 mM Cl⁻, and 28 mM lactate] or a similar volume of freshly-made Ringers ethyl pyruvate (EP) where EP (Sigma) was substituted for sodium lactate. A single dose was injected IP at 0, 6, or 12 hr after CLP surgery.

Serum levels of blood urea nitrogen (BUN), aspartate transaminase (AST), alanine transaminase (ALT), amylase, creatine kinase (CK) and lactate dehydrogenase (LDH) were measured using an autoanalyzer (Hitachi 917, Boehringer Mannheim, Indianapolis, MN). Serum creatinine were measured by a picric acid-based colorimetric kinetic autoanalyzer (Astra 8 autoanalyzer; Beckman Instruments, Fullerton, Calif.). Serum creatine was also measured by high-performance liquid chromatography (HPLC) Johns et al., B Biomed. Sci. Appl. 759:343 (2001)). Acetonitrile was added to serum, centrifuged, and the supernatant fraction was dried, resuspended in 5 mM sodium acetate pH 5.1, and chromatrographed isocratically on a PRP-X200 cation exchange column (Hamilton, Reno Nev.) and detected by UV absorbance at 234 nm (Agilent Technologies, Palo Alto, Calif.).

10% Formalin-fixed and paraffin-embedded kidney sections were stained with periodic acid-Schiff reagent (PAS) or naphthol AS-D chloroacetate esterase (Sigma; St. Louis, Mo.). Histological changes in the cortex and in the outer stripe of the outer medulla (OSOM) were assessed by quantitative measurements of tissue damage. Tubular damage was defined as tubular epithelial swelling, loss of brush border, vacuolar degeneration, necrotic tubules, cast formation, and desquamation. The degree of kidney damage was estimated at 400× magnification using 5 randomly selected fields for each animal by the following criteria: 0, normal; 1, areas of damage <25% of tubules; 2, damage involving 25-50% of tubules, 3, damage involving 50 to 75% of tubules; 4, 75-100% of the area being affected.

Cyr61 expression in the kidney was measured as described previously (Muramatsu, et. al., Kidney Int. 62:1601[2002]).

All data are expressed as means±SE. Differences between groups were examined for statistical significance by ANOVA with a multiple comparison correction (StatView 4.5, Berkeley, Calif.; or SigmaStat 2.0, SPSS, San Rafael, Calif.). A P value <0.05 was accepted as statistically significant.

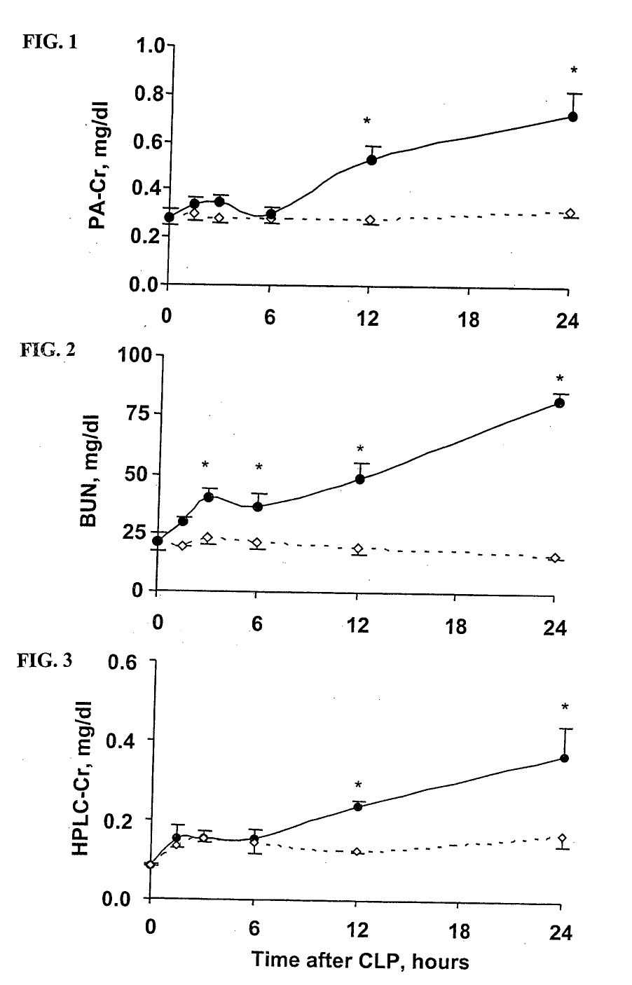

Cecal ligation puncture (CLP) sepsis caused time-dependent increases in markers of renal dysfunction (FIGS. 1-3). BUN was significantly increased as early as 3 hr after surgery, whereas the rise in creatinine was delayed until 12 and 24 hr after surgery.

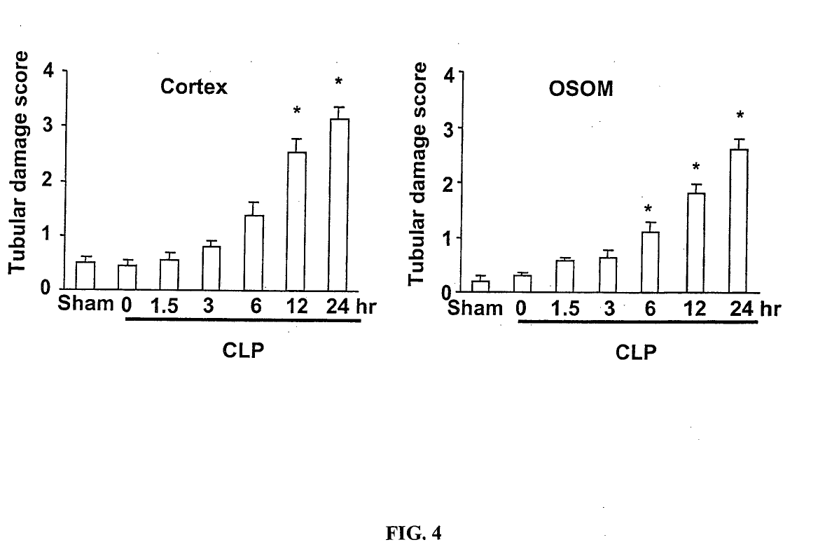

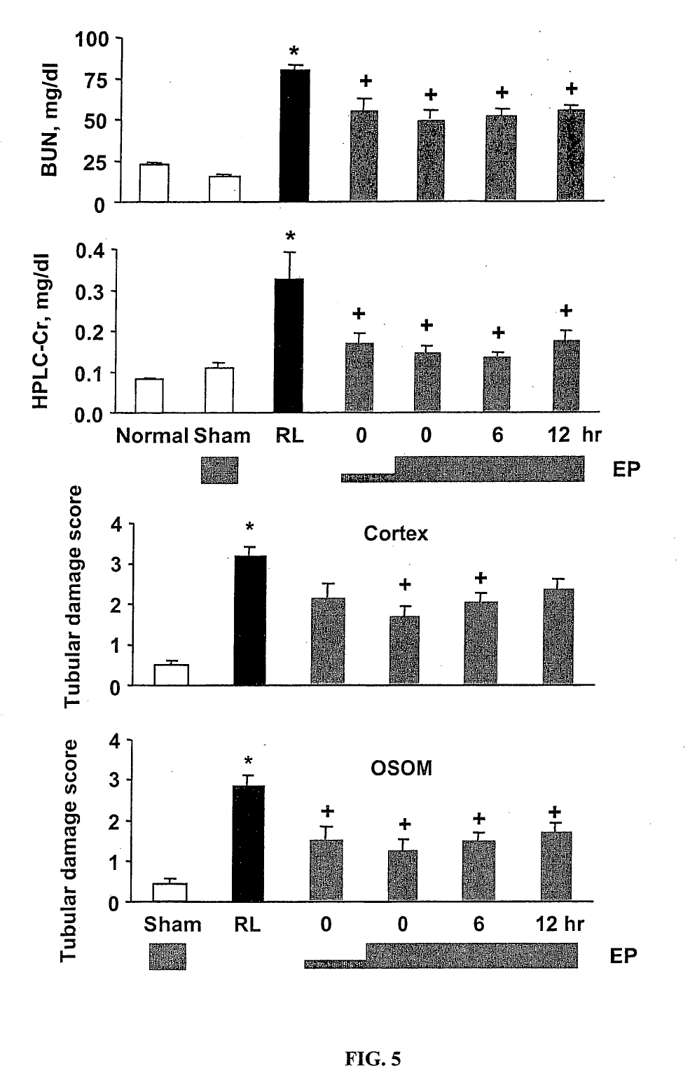

At 6 hr after CLP, histological examination of PAS stained-sections revealed focal tubular epithelial swelling, shortened brush border, and vacuolar degeneration in both the cortex and OSOM. At 12-24 hr after surgery, more extensive tubular damage was seen (FIG. 4) in both areas.

It was recently demonstrated that Cyr61 was rapidly induced in the kidney and secreted into the urine after ischemia reperfusion injury, but not after volume depletion (Muramatsu, et al., Kidney Int. 62:1601 [2002]). Therefore, Cyr61 was measured to investigate the timing of tubular injury in polymicrobial sepsis-induced acute renal failure. Cyr61 expression in the kidney was detected at 6 hr after surgery (a time at which serum creatinine was normal; FIG. 1), and sustained for at least 24 hr. Thus, renal injury occurs even before an increase in serum creatinine can be detected (FIGS. 1 and 3).

A single dose of either 8 or 40 mg/kg of EP after surgery significantly prevented the renal injury as measured by BUN or HPLC creatinine (FIG. 5; 8 mg/kg: 0.17±0.02 mg/dl; 40 mg/kg: 0.14±0.02 mg/dl vs. RL: 0.33±0.07 mg/dl). The higher dose of EP significantly reduced the renal injury even when delayed until 6 or 12 hr after surgery (HPLC creatinine: 6 hr: 0.13±0.01 mg/dl; 12 hr: 0.17±0.03 mg/dl). Administration of EP at either 0, 6, or 12 hr after CLP significantly reduced the tubular damage measured at 24 hr in both the cortex and OSOM (FIGS. 4 and 5).

EP still protected against renal injury even when treatment was started 12 hr after surgery. The renal injury was documented by suppression of sepsis-induced elevations of BUN, creatinine and tubular damage score, especially in the outer stripe of the outer medulla. The amount of protection was consistently 60-80% for tubular injury score, without much change with delayed treatment. This wide therapeutic window is a least 12 hr for sepsis-induced acute renal failure. Thus, EP can be a “rescue” therapeutic sepsis-induced renal injury. This prolonged window of opportunity may be important clinically because of the difficulty in the early detection of sepsis-induced ARF.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating acute renal failure in a subject, said method comprising the step of administering an effective amount of a composition comprising a an ester of a 2-ketoalkanoic acid or an amide of a 2-ketoalkanoic acid.

2. The method of claim 1 wherein the composition comprises an alkyl, aralkyl, alkoxyalkyl or carboxyalkyl ester of a 2-ketoalkanoic acid dissolved in a pharmaceutically acceptable carrier vehicle.

3. The method of claim 2 wherein the composition comprises an enolization agent.

4. The method of claim 3 wherein the enolization agent is a pharmaceutically acceptable inorganic, divalent cation.

5. The method of claim 1 wherein the composition comprises an ethyl, propyl, butyl, carboxymethyl, acetoxymethyl, carbethoxymethyl or ethoxymethyl ester of a 2-keto-butyrate, 2-ketopentanoate, 2-keto-3-methyl-butyrate, 2-keto-4-methyl-pentanoate or 2-keto-hexanoate.

6. The method of claim 2 wherein the subject is being treated prophylactically for acute renal failure.

7. The method of claim 6 wherein the subject is at risk for developing acute renal failure.

8. The method of claim 7 wherein the subject has at least two risk factors for developing acute renal failure.

9. The method of claim 7 wherein the subject has at least three risk factors for developing acute renal failure.

10. The method of claim 8 wherein the subject is undergoing contrast imaging.

11. The method of claim 8 wherein the subject has pre-existing renal disease/dysfunction.

12. The method of claim 8 wherein the subject is diabetic.

13. The method of claim 8 wherein the subject is being treated with nephrotoxic drugs.

14. The method of claim 8 wherein the subject is being treated for hypotension.

15. The method of claim 8 wherein the subject is being treated for hemorrhagic shock, systemic inflammation or sepsis.

16. The method of claim 8 wherein the subject is experiencing or likely to experience disruption of renal blood flow.

17. The method of claim 16 wherein the disruption of blood flow is due to surgery.

18. The method of claim 8 wherein the subject is experiencing liver failure or heart failure.

19. The method of claim 1 wherein the composition comprises pyruvamide.

20. The method of claim 2 wherein the composition comprises an ester of pyruvate.

21. A method of treating acute renal failure in a subject, said method comprising the step of administering an effective amount of a composition comprising ethyl pyruvate.

22. The method of claim 21 wherein the subject is being treated prophylactically for acute renal failure.

23. A method of prophylactically treating acute renal failure in a subject undergoing contrast imaging, said method comprising the step of administering an effective amount of a composition comprising an alkyl, aralkyl, alkoxyalkyl or carboxyalkyl ester of a 2-ketoalkanoic acid dissolved in a pharmaceutically acceptable carrier vehicle.

24. A method of prophylactically treating acute renal failure in a subject undergoing contrast imaging, said method comprising the step of administering an effective amount of ethyl pyruvate.

25. The method of claim 24 wherein the ethyl pyruvate is administered in a solution comprising between 0.1 M and 0.2 M lactate.

26. The method of claim 24 wherein the ethyl pyruvate is administered in a solution comprising between 105 mM and 110 mM NaCl and between 3.8 mM and 4.2 mM KCl.

27. The method of claim 1, wherein the subject is administered an ester of a 2-ketoalkanoic acid and wherein said ester is a glyceryl ester.

28. The method of claim 1, wherein said the subject is administered an ester of a 2-ketoalkanoic acid and wherein said ester is a ribosyl ester represented by the following formula: 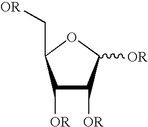
wherein each R is independently H, a 2-ketoalkanoate group or a C1-C3 acyl and at least one R is an 2-ketoalkanoate group.

29. The method of claim 1, wherein the subject is administered an ester of a 2-ketoalkanoic acid and wherein said ester is a glucosyl ester described by formulae (I) or (II): 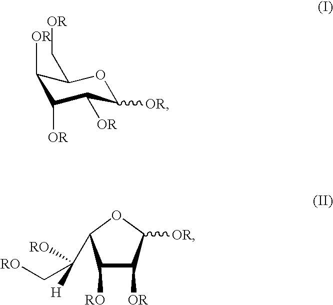
wherein each R is independently H, a 2-ketoalkanoate group or a C1-C3 acyl and at least one R is a 2-ketoalkanoate group.

30. The method of claim 1, wherein the subject is administered an ester of a 2-ketoalkanoic acid and wherein said ester is a dihydroxyacetone ester.
